(19) United States
(12) Patent Application Publication
(10) Pub. No.: US 2020/0052029 A1
(43) Pub. Date: Feb. 13, 2020

(54) ELECTRIC MODULE, ENDOSCOPE, AND METHOD FOR MANUFACTURING ELECTRIC MODULE

(71) Applicant: OLYMPUS CORPORATION, Tokyo (JP)

(72) Inventor: Shoichiro KAWAYOKE, Kamiina-gun (JP)

(73) Assignee: OLYMPUS CORPORATION, Tokyo (JP)

(21) Appl. No.: 16/657,522

(22) Filed: Oct. 18, 2019

Related U.S. Application Data
(63) Continuation of application No. PCT/JP2017/015984, filed on Apr. 21, 2017.

(51) Int. Cl. H01L 27/146; H01L 23/00; A61B 1/05; A61B 1/00

(52) U.S. Cl. CPC H01L 27/14636; H01L 27/14683; H01L 24/32; H01L 24/83; H01L 2224/83895; A61B 1/05; A61B 1/0011; H01L 2224/32147; H01L 24/94

(57) ABSTRACT

An electric module including a first member in which a plurality of first electrodes are arranged on a first face, a second member in which a plurality of second electrodes are arranged on a second face facing the first face, first resin for adhering the first face with the second face, and a bonding portion for electrically connecting each of the first electrodes and each of the second electrodes. The bonding portion is formed of an electroless plating membrane, and the first resin is arranged around the bonding portion.

### CROSS REFERENCE TO RELATED APPLICATION

This application is a continuation application of PCT/JP2017/015984 filed on Apr. 21, 2017, the entire contents of which are incorporated herein by this reference.

### BACKGROUND OF INVENTION

### 1. Field of the Invention

The present invention relates to an electric module, in which a plurality of first electrodes of a first member and a plurality of second electrodes of a second member are electrically connected to each other, an endoscope having the electric module, and a method for manufacturing the electric module.

### 2. Description of the Related Art

An electric module including an image pickup device such as a CCD is arranged in an endoscope at a distal end portion of an elongated flexible insertion portion.

Japanese Patent Application Laid-Open Publication No. 2008-42169 discloses a method for bonding electrodes of two semiconductor chips laminated using an adhesive layer by arranging a plating membrane on a wall of the adhesive layer.

### SUMMARY OF THE INVENTION

An electric module in an embodiment of the present invention comprises a first member in which a plurality of first electrodes are arranged on a first face, a second member in which a plurality of second electrodes are arranged on a second face facing the first face, first resin for adhering the first face with the second face, and a bonding portion for electrically connecting each of the first electrodes and each of the second electrodes. The bonding portion is formed of an electroless plating membrane, and the first resin is arranged around the bonding portion.

An endoscope in another embodiment has an electric module, and the electric module comprises a first member in which a plurality of first electrodes are arranged on a first face, a second member in which a plurality of second electrodes are arranged on a second face facing the first face, first resin for adhering the first face with the second face, and a bonding portion for electrically connecting each of the first electrodes and each of the second electrodes. The bonding portion is formed of an electroless plating membrane, and the first resin is arranged around the bonding portion.

Furthermore, a method for manufacturing an electric module in another embodiment comprises a manufacturing step of manufacturing a first member in which a plurality of first electrodes are arranged on a first face, and a second member in which a plurality of second electrodes are arranged on a second face facing the first face, an adhesion step of adhering the first member with the second member using first resin patterned not to cover the plurality of first electrodes and the plurality of second electrodes by arranging the first member and the second member so that the first face and the second face face each other, and a plating step of electrically connecting each of the plurality of first electrodes and each of the plurality of second electrodes to each other by a bonding portion formed of an electroless plating membrane.

### BRIEF DESCRIPTION OF THE DRAWINGS

FIG. 1 is a cross-section view of an electric module in a first embodiment;

FIG. 2 is a cross-section view along a II-II line of FIG. 1 of the electric module in the first embodiment;

FIG. 3 is an exploded view of the electric module in the first embodiment;

FIG. 4 is a flowchart for explaining a method for manufacturing an electric module in the first embodiment;

FIG. 5 is a perspective view for explaining the method for manufacturing the electric module in the first embodiment;

FIG. 6 is a cross-section view for explaining the method for manufacturing the electric module in the first embodiment;

FIG. 7 is a cross-section view for explaining the method for manufacturing the electric module in the first embodiment;

FIG. 8A is a cross-section view for explaining the method for manufacturing the electric module in the first embodiment;

FIG. 8B is a cross-section view for explaining the method for manufacturing the electric module in the first embodiment;

FIG. 8C is a cross-section view for explaining the method for manufacturing the electric module in the first embodiment;

FIG. 9 is a cross-section view of an electric module in a modification example 1 of the first embodiment;

FIG. 10 is a cross-section view of an electric module in a modification example 2 of the first embodiment;

FIG. 11 is a cross-section view of an electric module in a modification example 3 of the first embodiment;

FIG. 12A is a cross-section view of an electric module in a second embodiment;

FIG. 12B is a cross-section view along a line XIB-XIB of FIG. 12A of the electric module in the second embodiment;

FIG. 13A is a cross-section for explaining a method for manufacturing an electric module in the second embodiment;

FIG. 13B is a cross-section for explaining a method for manufacturing an electric module in the second embodiment; and

FIG. 14 is a perspective view of an endoscope system including an endoscope in a third embodiment.

### DETAILED DESCRIPTION OF THE PREFERRED EMBODIMENTS

### First Embodiment

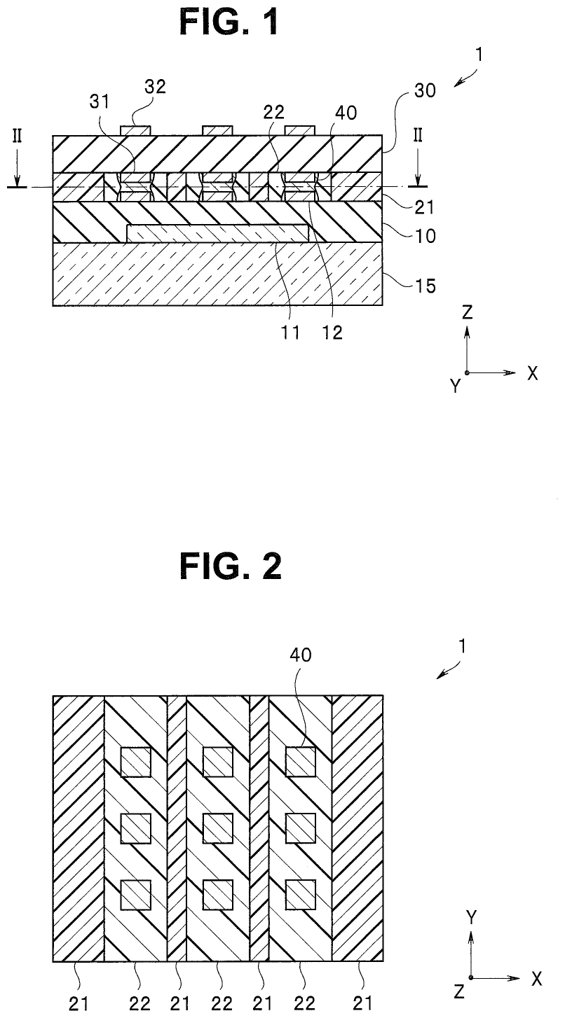

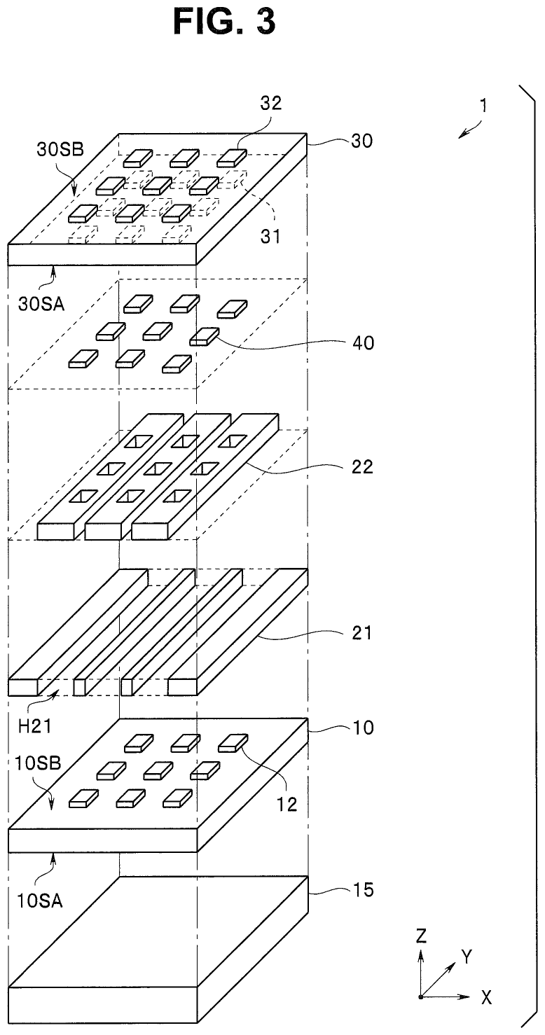

As shown in FIG. 1 to FIG. 3, an electric module 1 in this embodiment includes an image pickup device 10 that is a first member and that is adhered to a cover glass 15, and a semiconductor element 30 that is a second member.

In the following explanation, the drawings in each embodiment are merely schematic, and so it should be noted that a relationship between thickness and width of each portion, a ratio of the thickness of each portion, and the like are different from actual ones, and portions with a relationship or a ratio different from each other may be included in the drawings. Illustration and numbering of some constituting elements may be omitted.

The image pickup device 10 is a parallel plate-like element having a light receiving face 10SA and a first face 10SB facing the light receiving face 10SA. A plurality of first electrodes 12 are two-dimensionally arranged on the first face 10SB of the image pickup device 10.

The image pickup device 10 is a CMOS (complementary metal oxide semiconductor) image sensor, a CCD (charge coupled device), or the like. Image pickup signals outputted from a light receiving portion 11 are transmitted to the first electrodes 12 via a through wiring (not shown). The image pickup device 10 may be any of an FSI (front side illumination) type image sensor or BSI (back side illumination) type image sensor.

The cover glass 15 protects the light receiving portion 11 of the image pickup device 10, but is not a necessary constituting element of the electric module 1. On the contrary, not only the cover glass 15, but also a plurality of optical members such as a plurality of lenses may be arranged on the light receiving face 10SA of the image pickup device 10.

On the other hand, the semiconductor element 30 is a parallel plate-like element having a second face 30SA and a third face 30SB facing the second face. A plurality of second electrodes 31 are two-dimensionally arranged on the second face 30SA of the semiconductor element 30. Also, a plurality of third electrodes 32 are two-dimensionally arranged on the third face 30SB. The second electrodes 31 and the third electrodes 32 are electrically connected via a through wiring (not shown).

A circuit for primary-processing the pickup signals outputted from the image pickup device 10 and processing control signals controlling the image pickup device 10 is formed on the semiconductor element 30. For example, the semiconductor element 30 includes an AD conversion circuit, a memory, a transfer output circuit, a filter circuit, a thin membrane capacitor, and a thin membrane inductor.

The first face 10SB of the image pickup device 10 and the second face 30SA of the semiconductor element 30 face and are adhered to each other using first resin 21. An interval between the first face 10SB and the second face 30SA is identical to thickness of the first resin 21. The plurality of first electrodes 12 and the plurality of second electrodes 31 are electrically connected to each other by a bonding portion 40 formed of an electroless plating membrane. The first resin 21 is patterned under a condition not to cover the bonding portion 40, so it is arranged around the bonding portion 40.

The electroless plating membrane of the bonding portion 40 is made of conductive metal mainly composed of copper, gold, nickel or the like. At least surface of the first electrodes 12 and the second electrodes 31 are covered by metal serving as a catalyst of an electroless plating reaction. The electroless plating membrane is selectively precipitated only on the surfaces of the first electrodes 12 and the second electrodes 31, and connect the first electrodes 12 and the second electrodes 31.

A plurality of through holes H21 whose opposite end faces are both open are provided on the first resin 21. In other words, the through hole 1121 has a lower face that is a first face 10SB, an upper face that is a second face 30SA, and a side face that is first resin 21. An inside of the through hole H21 in which the bonding portion 40 is arranged is filled with the second resin 22.

An electric member with poor heat resistance may deteriorate reliability when solder bonding is used. However, the image pickup device 10 and the semiconductor element 30 are electrically connected to each other by a bonding portion 40 made of the electroless plating membrane formed by a low temperature process at less than 100° C. Therefore, there is no risk that the electric module 1 is damaged by heat at the time of bonding, so the electric module has high reliability.

Also, when a plurality of electrodes are arranged at narrow pitches, the adjacent electrodes may be short-circuited at the time of bonding. However, the electroless plating membrane is selectively precipitated only to the surfaces of the first electrodes 12 and the second electrodes 31, so there is no risk that the adjacent electrodes are short-circuited.

Furthermore, even when the first face 10SB and the second face 30SA are not completely parallel to each other, the plurality of first electrodes 12 and the plurality of second electrodes 31 are connected to each other by the respective bonding portions 40. Even when the first electrodes 12 and the second electrodes 31 facing each other are slightly displaced from each other, they are connected by the bonding portion 40.

In the electric module 1, the first member is the image pickup device 10, and the second member is an image pickup module of the semiconductor element 30. However, the first member is not limited to an image pickup device. Also, the second member is not limited to a semiconductor element and, for example, may be an interposer made of silicon.

### Method for Manufacturing Electric Module

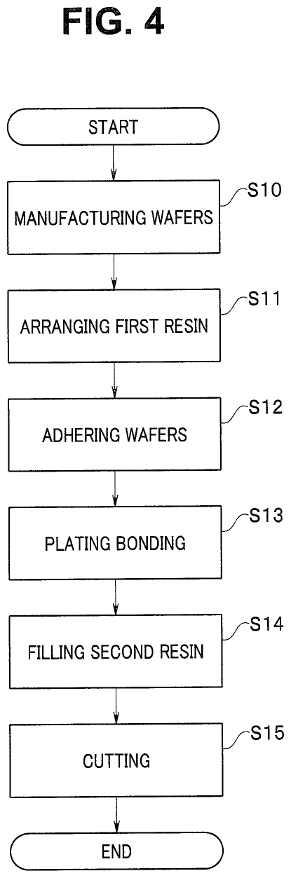

Then, a method for manufacturing an electric module 1 will be explained with reference to a flowchart of FIG. 4.

### <Step S10> Wafer Manufacturing Step

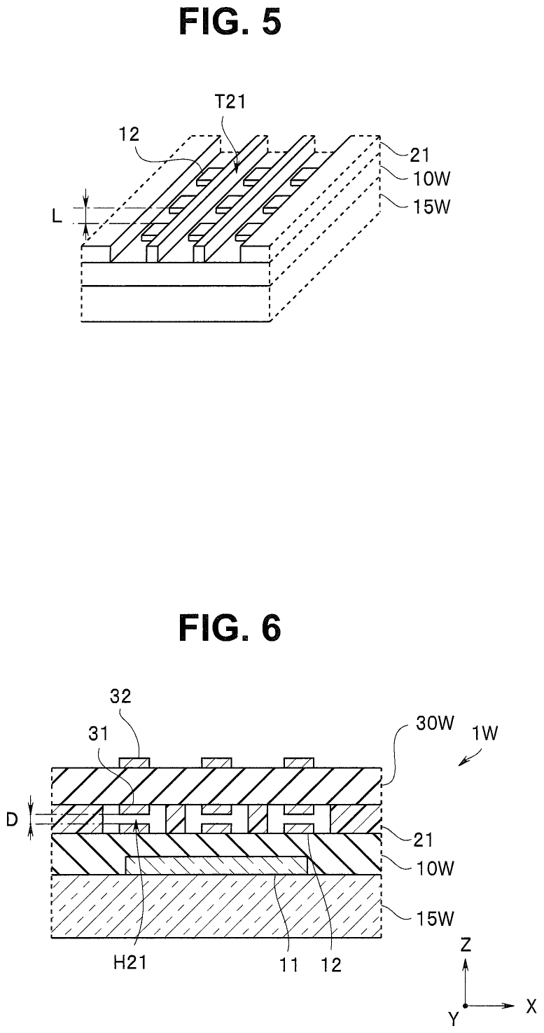

An image pickup wafer 10W (see FIG. 5) including the plurality of image pickup devices 10 and an element wafer 30W (see FIG. 6) including the plurality of semiconductor elements 30 are manufactured.

For example, the image pickup wafer 10W on which a plurality of light receiving portions 11 and the like are arranged is manufactured using a semiconductor manufacturing technique well known to a silicon wafer. A peripheral circuit for primary-processing output signals of the light receiving portions 11 and processing drive control signals may be formed on the image pickup wafer 10W.

To protect the light receiving portions 11 of the image pickup wafer 10W, a cover glass wafer 15W made of plane glass (see FIG. 6) is adhered. Then, after grinding/polishing processing is performed from the first face 10SB of the image pickup wafer 10W, a plurality of through wirings (not shown) and the plurality of first electrodes 12 connected to the light receiving portions 11 are arranged. When the through wirings and the first electrodes 12 are connected via a surface wiring of the first face 10SB, the arrangement of the plurality of through wirings may be different from the arrangement of the plurality of first electrodes 12.

A plurality of semiconductor circuits and the like are arranged on the element wafer 30W using a semiconductor manufacturing technique well known regarding a silicon wafer and the like, and the plurality of second electrodes 31 are arranged on the second face 30SA. The plurality of third electrodes 32 are arranged on the third face 30SB facing to the second face 30SA.

The plurality of first electrodes 12 and the second electrodes 31 face each other, when the first face 10SB and the second face 30SA face each other. Also, the first electrodes 12 and the second electrodes 31 are rectangular, but may be circular.

It is preferable that at least surfaces of the first electrodes 12 and the second electrodes 31 are covered by metal serving as a catalyst of an electroless plating reaction. For example, the first electrodes 12 and the second electrodes 31 are made of metal the same as electroless plated metal such as copper, gold, palladium, nickel and the like, or more electrochemically noble metal than the electroless plated metal, i.e., metal with high standard oxidation-reduction potential.

When immersed with electroless plating liquid, by a substitution reaction, metal ions of the plating liquid may be a metal membrane and a substitution metal membrane may be a catalyst. When the substitution metal membrane is a catalyst, the first electrodes 12 and the second electrodes 31 may be less electrochemically noble metal than the electroless plated metal.

### <Step S11> First Resin Arranging Step

As shown in FIG. 5, the first resin 21 is arranged on the first face 10SB of the image pickup wafer 10W, so as not to cover the plurality of first electrodes 12. For example, the first resin 21 that is photosensitive adhesive is arranged entirely on the first face 10SB, and patterned by a photolithography process, so as to form a groove T21. In other words, the groove T21 is formed by patterning under a condition that the first resin 21 does not cover the bonding portion 40, i.e., a condition that it is arranged around the bonding portion 40.

The plurality of first electrodes 12 are two-dimensionally arranged, and the first resin 21 is divided by the plurality of grooves T21, so that the plurality of first electrodes 12 are exposed. The groove T21 has a bottom face that is the first face 10SB on which the plurality of first electrodes 12 are arranged, and both side faces made of the first resin 21, and is inserted to an outer periphery of the image pickup wafer 10W. The first resin 21 may be patterned by a printing method or an ink jet method.

In the image pickup wafer 10W shown in FIG. 5, the plurality of first electrodes 12 linearly aligned are exposed to the bottom face of one groove T21. However, the plurality of first electrodes 12 exposed to the bottom face of one groove T21 do not have to be linearly aligned.

Also, the first resin 21 may be arranged on the second face 30SA of the element wafer 30W, and may be arranged on the image pickup wafer 10W and the element wafer 30W, respectively. The plurality of first electrodes 12 are aligned at intervals L (see FIG. 5).

### <Step S12> Wafer Adhesion Step

The first face 10SB of the image pickup wafer 10W and the second face 30SA of the element wafer 30W are adhered using the first resin 21, for example, by a thermal compression bonding method, so as to manufacture a bonding wafer 1W. An interval between the first face 10SB of the bonding wafer 1W and the second face 30SA of the element wafer 30W is defined by thickness of the first resin 21.

The groove T21 of the first resin 21 is made a through hole H21 of the bonding wafer 1W by adhesion of the element wafer 30W. In other words, the groove T21 is covered by the second face 30SA on its top face, and becomes a through hole H21.

An interval D between the first electrode 12 and the second electrode 31 of the image pickup wafer 10W facing each other is smaller than an interval L of the plurality of first electrodes 12 (see FIG. 5), and is preferably set to less than a half of the interval L, for example, 10 μm or less. The interval D between the first electrode 12 and the second electrode 31 may be zero, i.e., the first and second electrodes may be in contact with each other.

### <Step S13> Plating Step

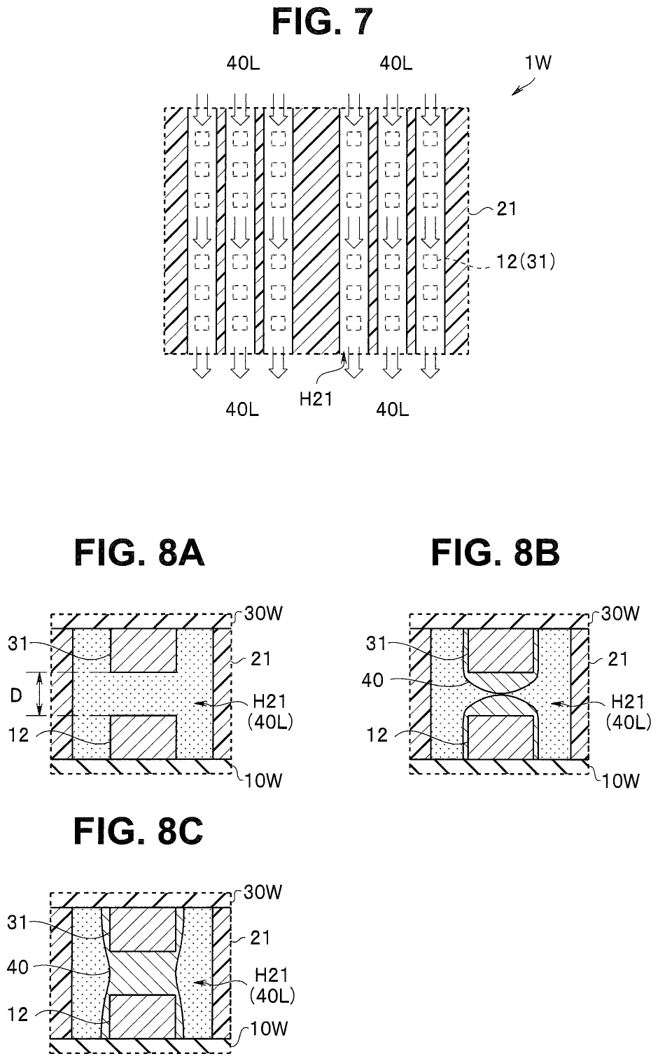

As shown in FIG. 7 and FIG. 8A, electroless plating liquid 40L flows into the through hole H21 of the bonding wafer 1W, inside of which the first electrode 12 and the second electrode 31 facing each other are arranged.

The electroless plating liquid 40L starts a precipitation reaction by a reduction reaction of a reducing agent using more electrochemically noble metal than contained metal as a catalyst, and further continues precipitation by a self-catalysis reaction using the precipitated metal as a catalyst. Therefore, as shown in FIG. 8B, the plating membrane is selectively precipitated only on the surface of the first electrode 12 and the surface of the second electrode 31, and isotopically grows.

Electroless copper plating liquid with formalin as a reduction agent, electroless gold plating liquid with DMAB and the like as a reduction agent, electroless nickel plating liquid with hypophosphorous acid, DMAB or the like as a reduction agent, and the like are used as the electroless plating liquid 40L. Also, it is preferable to use low phosphorous type NiP base plating liquid with a small phosphorous content, or Nib base plating liquid with DMAB and the like as a reduction agent, by which a plating membrane with low electric resistance is deposited, as electroless nickel plating liquid. In other words, the bonding portion 40 is made of Cu, Au, Ni, Nib alloy, NiP alloy or the like.

As shown in FIG. 8C, when the thickness of the plating membrane of the bonding portion 40 becomes 0.5 D or more, the first electrode 12 and the second electrode 31 are bonded to each other. For example, when the interval D between the first electrode 12 and the second electrode 31 is 10 μm, if the thickness of the plating membrane becomes 5 μm or more, the first electrode 12 and the second electrode 31 are bonded to each other.

The first electrode 12 and the second electrode 31 are plane electrodes, but are more preferable to be curve-shaped concave electrodes, because a void is rarely formed at the bonding portion 40.

A flow of the electroless plating liquid 40L is controlled, for example, by a pump so that the electroless plating liquid 40L flows into the through hole H21 from one opening of the through hole H21, i.e., one direction of the outer periphery of the bonding wafer 1W (top in FIG. 7), and flows out of the other opening (bottom in FIG. 7). Therefore, inside of the through hole H21 is always filled with fresh electroless plating liquid 40L.

It is preferable that an orientation of the flow of the electroless plating liquid 40L is reversed some times during plating. The flow of the electroless plating liquid 40L may be formed by vibration of the bonding wafer 1W immersed with the electroless plating liquid 40L. In other words, in a plating step, it is preferable that the electroless plating liquid 40L flows alternately from openings at both ends of the through hole H21, so as to make the thicknesses of the plurality of bonding portions 40 uniform.

The electroless plating liquid 40L is selected according to metal of the first electrode 12 and the second electrode 31. In other words, the metal of the first electrode 12 and the second electrode 31 are selected according to the metal of the bonding portion 40 deposited by the electroless plating liquid 40L. For example, when the bonding portion 40 is gold, the first electrode 12 and the second electrode 31 are covered with gold. Even when the electrodes are made of less noble metal such as copper than gold, it is good enough to cover the surface with gold. When the bonding portion 40 is copper, gold that is more electrochemically noble metal than copper, or the like is selected for the first electrode 12 and the second electrode 31. When the bonding portion 40 is nickel, the first electrode 12 and the second electrode 31 are covered with nickel, gold that is more electrochemically noble metal than nickel, or the like.

The electroless plating liquid 40L is aqueous solution containing metal ions and a reduction agent, and temperature is about 90° C. from room temperature lower than solder melting temperature. Therefore, the image pickup device 10 and the semiconductor element 30 are not damaged by plating treatment.

It is preferable that the bonding wafer 1W is pre-treated before the plating treatment. For example, when the first electrode 12 and the second electrode 31 are copper, an oxidized membrane on the surface is removed by acid pre-treatment liquid. When the first electrode 12 and the second electrode 31 are aluminum, zinc is arranged on the surface by zincate treatment. An electroless nickel membrane is precipitated on zincate-treated aluminum, using a substitution plated nickel membrane as a catalyst.

### <Step S14> Second Resin Filling Step

After a plating step (S13), liquid-like uncured second resin 22 is injected to the through hole H21, and cured.

The second resin 22 does not have to be filled. After a cutting step (S15), the second resin 22 may be injected.

### <Step S15> Cutting Step (Dicing Step)

The electric module 1 is manufactured by cutting processing of the bonding wafer 1W.

In a method for manufacturing an electric module 1, the bonding portion 40 is arranged by an electroless plating method that is a low temperature process at lower than 100° C. In the method for manufacturing the electric module 1, there is no risk of damage due to heat, so the electric module 1 has high reliability. Also, the electroless plating membrane is selectively precipitated only on the surfaces of the first electrode 12 and the second electrode 31, there is no risk of short-circuiting the adjacent electrodes. Furthermore, even when the first face 10SB and the second face 30SA are not completely parallel to each other, the first electrode 12 and the second electrode 31 can be connected to each other by the respective bonding portions 40.

### Modification of First Embodiment

The electric module in modification of the first embodiment is similar to the electric module 1 and has the same effects, so same symbols are added to constitution elements with the same function, and explanation thereof will be omitted.

### Modification 1 of First Embodiment

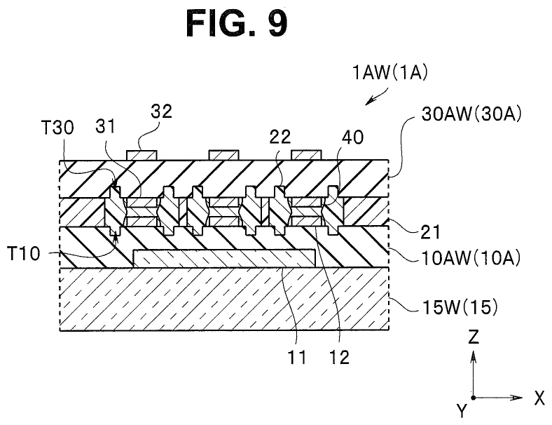

As shown in FIG. 9, a bonding wafer 1AW (electric module 1A) in the present modification has a groove T10 communicative with the through hole H21 on the first face 10SB of an image pickup wafer 10AW (image pickup device 10A), and a groove T30 communicative with the through hole 1121 on the second face 30SA of the element wafer 30W (semiconductor element 30A).

When the interval D between the first face 10SB of the image pickup wafer 10W and the second face 30SA of the element wafer 30W is narrow, a flow path of the electroless plating liquid 40L is narrow, so it is not easy to flow the electroless plating liquid 40L. The groove of the electric module 1A serves as a main flow path of the electroless plating liquid 40L, and fresh electroless plating liquid 40L is always supplied to inside of the through hole H21. Therefore, an electroless plating membrane can be stably deposited.

Needless to say, the groove communicative with the through hole H21 has the same effects as the electric module 1A, at least on any of the first face 10SB of the image pickup wafer 10W or the second face 30SA of the element wafer 30W.

### Modification 2 of First Embodiment

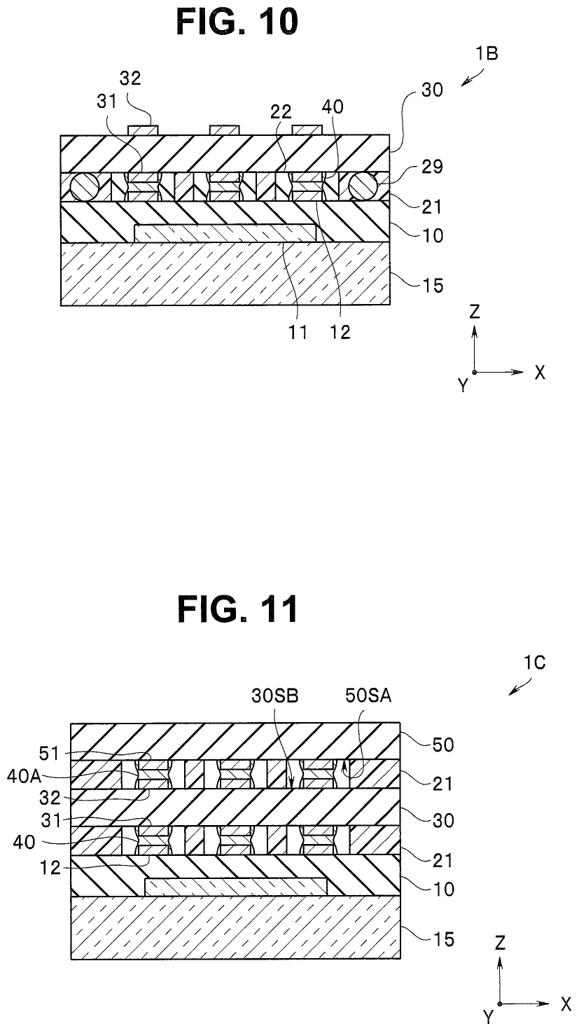

As shown in FIG. 10, an electric module 1B in the present modification has an interval defining member 29 for defining an interval between the first face 10SB and the second face 30SA, i.e., an interval D between the first electrode 12 and the second electrode 31.

The interval defining member 29 is composed of a hard material that can define the interval D, in the wafer adhesion step (S12) for adhering the image pickup wafer 10W with the element wafer 30W using the first resin 21 to manufacture the bonding wafer 1W.

For example, the interval defining member 29 has a sphere shape or a rod-like shape, and may be a convex portion of the image pickup wafer 10W or the element wafer 30W. Furthermore, at least three interval defining members 29 in the case of the sphere shape, and at least two interval defining members 29 in the case of the rod-like shape only have to be arranged. In other words, there is no need to arrange the interval defining member 29 on all of the electric modules 1B manufactured from the bonding wafer 1W by cutting. The interval defining member 29 may be arranged at an outer periphery that does not become an electric module 1B of the bonding wafer 1W.

The interval D of the electric module 1B is precisely and simply defined by the interval defining member 29. Therefore, it is easier to manufacture the electric module 1B than to manufacture the electric module 1, and a yield is high.

### Modification 3 of First Embodiment

As shown in FIG. 11, an electric module 1C in the present modification further includes a second semiconductor element 50 that is a third member. The second semiconductor element 50 is a parallel plate-like element having a fourth face 50SA and an upper face 50SB facing the fourth face 50SA.

A plurality of fourth electrodes 51 are two-dimensionally arranged on the fourth face 50SA of the semiconductor element 50. The plurality of third electrodes 32 and the plurality of fourth electrodes 51 are electrically connected to each other by the respective bonding portions 40A made of the electroless plating membrane.

The second semiconductor element 50 is bonded in the wafer bonding step (S12), and the bonding portion 40A is arranged at the same time and in the same plate-bonding step (S13) as the bonding portion 40 for bonding the image pickup device 10 and the first semiconductor element 30.

Also, a plurality of third electrodes 32 and a plurality of fourth electrodes 51 are aligned in the same manner, but the plurality of first electrodes 12 and the plurality of second electrodes 13 may be aligned in a different manner. The number of the plurality of third electrodes 32 and the plurality of fourth electrodes 51 may be different from the number of the plurality of first electrodes 12 and the plurality of second electrodes 31. However, it is preferable that the interval between the third electrodes 32 and the fourth electrodes 51 is identical to the interval D between the first electrodes 12 and the second electrodes 31.

An electric module 1C has a second semiconductor element 50 that is a third member, so it has a higher performance and is easier to miniaturize than the electric module 1. In a method for manufacturing an electric module 1C, a plurality of wafer bonding portions can be arranged at the same time, so that manufacturing the electric module is easy. The electric module 1C may have an interval defining member 29 as is similar to the electric module 1B.

The electric module 1C may further have a third semiconductor element electrically connected by a bonding portion made of an electroless plating membrane, on the second semiconductor element 50 that is a third member. In other words, an element laminate body including a plurality of semiconductor elements may be bonded on the first face 10SB of the image pickup device 10.

The image pickup modules 1B, 1C have the same effects as the image pickup module 1A, if the image pickup modules 1B, 1C have a groove T10 and the like serving as a flow path, like the image pickup module 1A.

### Second Embodiment

An electric module 1D in a second embodiment is similar to the electric module 1 and the like, so constitution elements with the same function have the same symbols and explanation thereof will be omitted.

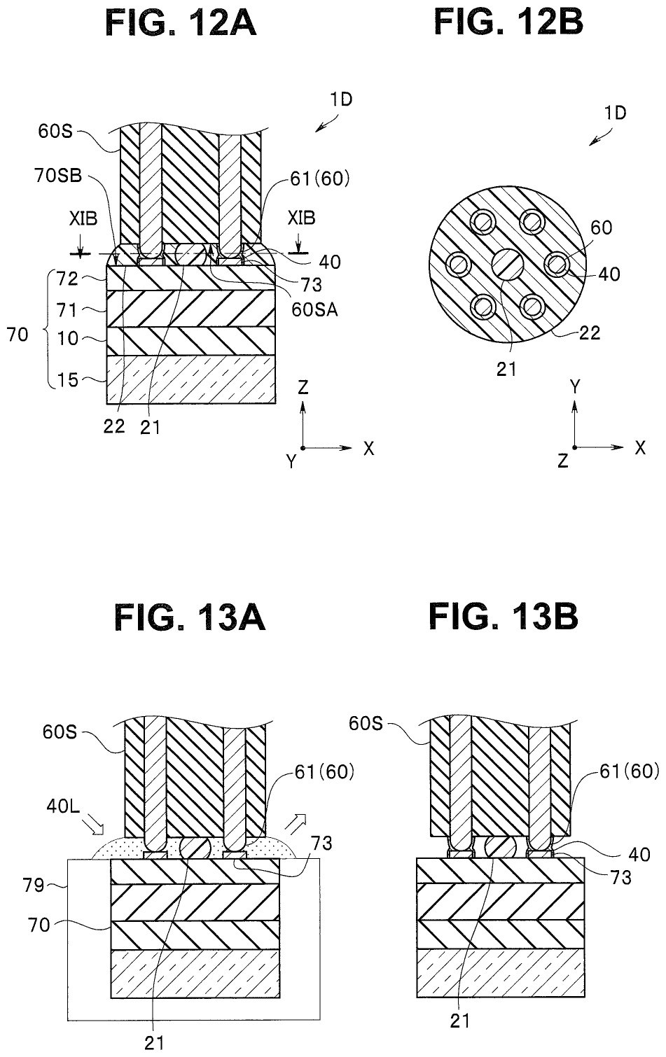

As shown in FIG. 12A and FIG. 12B, in the electric module 1D, the second member is a complex cable 60S having a plurality of cables 60. Second electrodes 61 that are distal end portions of the plurality of cables 60 including conductors are two-dimensionally arranged on the second face 60SA. The distal end portions of the cables 60 made of copper may be covered with gold.

The first member is a wafer level chip size and package type laminate element 70, on which the cover glass 15, the image pickup device 10, a first semiconductor element 71 and a second semiconductor element 72. The first semiconductor element 71 and the second semiconductor element 72 primary-process image pickup signals outputted by the image pickup device 10, and process control signals controlling the image pickup device 10, as are similar to the semiconductor elements 30, 50.

A plurality of first electrodes 73 are two-dimensionally arranged on a first face 72SB that is a rear face of a second semiconductor element 72 at a rearmost portion of the laminate element 70.

The first face 72SB and the second face 60SA are adhered using the first resin 21. Then, the plurality of first electrodes 73 and the plurality of second electrodes 61 are respectively connected electrically to each other by the respective bonding portions 40 made of the electroless plating membrane. Each of the bonding portion 40 is sealed by the second resin 22.

As shown in FIG. 13A, the complex cable 60S and the laminate element 70 are adhered using the first resin 21. The laminate element 70 is covered with a protection member 79 except for the second face 60SA. In the electric module 1D, when the complex cable 60S and the laminate element 70 are adhered to each other, the interval D between the first electrode 12 and the second electrode 31 is zero. However, the first electrode 73 and the second electrode 61 are merely in contact with each other, and are not connected electrically stably. Of course, the first electrode 12 and the second electrode 31 may face each other in a condition of a predetermined interval D.

The electroless plating liquid 40L flows to a space between the first face 72SB and the second face 60SA around the first resin 21. Then, as shown in FIG. 13B, the first electrode 73 and the second electrode 61 are bonded to each other by the bonding portion 40 made of the electroless plating membrane, and is connected electrically stably. Finally, the bonding portion 40 is sealed by the second resin 22.

In the electric module 1D, a through hole serving as a flow path of the electroless plating liquid may be formed by the patterned first resin 21.

As explained above, the electric module 1D has the same effects as the electric module 1, while the second member is the complex cable 60S having the plurality of cables 60, tips of which are second electrodes 61.

### Third Embodiment

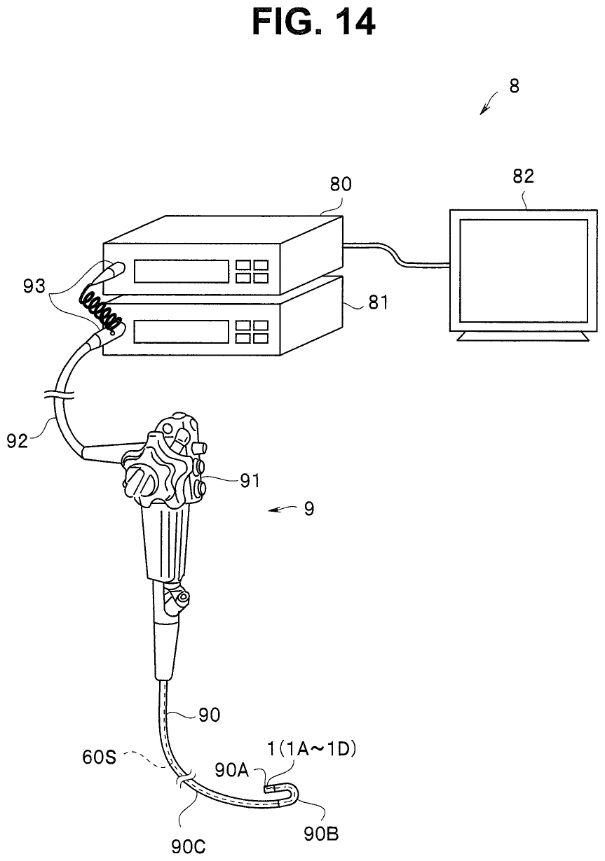

As shown in FIG. 14, an endoscope system 8 including an endoscope 9 in the present embodiment includes the endoscope 9, a processor 80, a light source device 81, and a monitor 82. The endoscope 9 has an insertion portion 90, an operation portion 91, and a universal code 92. An insertion portion 90 of the endoscope 9 is inserted in a body cavity of a subject, and the endoscope 9 takes an image in a body of the subject and outputs the image signals.

The insertion portion 90 is constituted by a distal end portion 90A at which the electric modules 1, 1A to 1D (hereinafter referred to as the electric module 1 and the like) are arranged, a curve portion 90B that is freely curved and is connected with a base end side of the distal end portion 90A, and a soft portion 90C connected with a base end side of the curve portion 90B. The curve portion 90B is curved by an operation of an operation portion 91. Also, the endoscope 9 may be a hard mirror, or a capsule type endoscope.

The operation portion 91 provided with various buttons for operating the endoscope 9 is arranged at a base end side of the insertion portion 90 of the endoscope 9.

The light source device 81 has, for example, a white LED. Illumination lights emitted by the light source device 81 are guided to the distal end portion 90A via a light guide (not shown) inserted through the universal code 92 and the insertion portion 90, so as to illuminate an object.

The endoscope 9 has an insertion portion 90, an operation portion 91, and a universal code 92, and transmits image pickup signals outputted by the electric module 1 and the like arranged at the distal end portion 90A of the insertion portion 90 via a complex cable 60S inserted at the insertion portion 90.

The electric module 1 and the like are very small and have high reliability, so that the distal end portion 90A at the insertion portion 90 of the endoscope 9 has a small diameter and high reliability. The endoscope 9 is easy to manufacture.

The present invention is not limited to the above-mentioned embodiment and variations, but various changes, combinations and applications can be made without departing from the gist of the invention.

What is claimed is:

1. An electric module comprising:
a first member in which a plurality of first electrodes are arranged on a first face,
a second member in which a plurality of second electrodes are arranged on a second face facing the first face,
first resin for adhering the first face with the second face, and
a bonding portion for electrically connecting each of the first electrodes and each of the second electrodes,
wherein the bonding portion is formed of an electroless plating membrane, and the first resin is arranged around the bonding portion.

2. The electric module according to claim 1, comprising an interval defining member for defining an interval between the first face and the second face.

3. The electric module according to claim 1, wherein the first member and the second member are parallel plate-like elements, and a through hole in which the bonding portion is arranged, both end faces of the through hole being open, is provided in the first resin.

4. The electric module according to claim 3, wherein a groove communicating with the through hole is provided on at least one of the first face and the second face.

5. The electric module according to claim 3, further comprising second resin filling the through hole.

6. The electric module according to claim 3, wherein the second member has a third face facing the second face and a plurality of third electrodes are arranged on the third face,
the electric module comprises a third member that is a parallel plate-like element and has a fourth face on which a plurality of fourth electrodes are arranged, and the first resin for adhering the third face with the fourth face, and
each of the plurality of third electrodes and each of the plurality of fourth electrodes are electrically connected to each other by a bonding portion made of the electroless plating membrane.

7. The electric module according to claim 1, wherein the second member is a complex cable having a plurality of cables, tip ends of which are the second electrodes.

8. The electric module according to claim 1, wherein the first member is an image pickup device having a light receiving portion on a light receiving face facing the first face.

9. An endoscope comprising an electric module, the electric module including:
a first member in which a plurality of first electrodes are arranged on a first face,
a second member in which a plurality of second electrodes are arranged on a second face facing the first face,
first resin for adhering the first face with the second face, and
a bonding portion for electrically connecting each of the first electrodes and each of the second electrodes,
wherein the bonding portion is formed of an electroless plating membrane, and the first resin is arranged around the bonding portion.

10. A method for manufacturing an electric module, the method comprising:
manufacturing a first member on which a plurality of first electrodes are arranged on a first face, and a second member on which a plurality of second electrodes are arranged on a second face;
adhering the first member with the second member using first resin arranging the first face and the second face facing each other, and patterned not to cover the plurality of first electrodes and the plurality of second electrodes; and
electrically connecting each of the plurality of first electrodes and each of the plurality of second electrodes to each other by a bonding portion made of an electroless plating membrane.

11. The method for manufacturing the electric module according to claim 10,
wherein when the first member and the second member are adhered, a first wafer including the plurality of first members and a second wafer including the plurality of second members are adhered by the first resin having a groove, and the groove is made a through hole, inside of which the first electrodes and the second electrodes are arranged,
when connected by the electroless plating membrane, electroless plating liquid flows into the through hole, and
after being connected by the electroless plating member, the module is diced into a plurality of electric modules.

12. The method for manufacturing the electric module according to claim 11, wherein when connected by the electroless plating membrane, the electroless plating liquid alternately flows from openings at both ends of the through hole.

13. The method for manufacturing the electric module according to claim 11, wherein after being connected by the electroless plating membrane, the through hole is filled with second resin.

14. The method for manufacturing the electric module according to claim 10, wherein the first member is an image pickup device having a light receiving portion on a light receiving face facing the first face.
